(19) United States
(12) Patent Application Publication
(10) Pub. No.: US 2017/0274017 A1
(43) Pub. Date: Sep. 28, 2017

(54) BONE FRACTURE HEALING WITH PRIMED DISTAL BONE MARROW MESENCHYMAL STROMAL CELLS

(71) Applicants: Smith & Nephew, Inc., Memphis, TN (US); University of Twente, Enschede (NL)

(72) Inventors: Chen-rei Wan, Arlington, MA (US); Susan Hsiong, Winchester, MA (US); Ada Au, Lexington, MA (US); Corina Adriana Ghebes, Enschede (NL)

(73) Assignees: Smith & Nephew, Inc., Memphis, TN (US); University of Twente, Enschede (NL)

(21) Appl. No.: 15/504,797

(22) PCT Filed: Aug. 21, 2015
(86) PCT No.: PCT/US2015/046360

§ 371 Date: Feb. 17, 2017

Related U.S. Application Data
(60) Provisional application No. 62/040,222, filed on Aug. 21, 2014.

(51) Int. Cl. A61K 35/28; A61L 27/38; C12N 5/0775

(52) U.S. Cl. CPC A61K 35/28; C12N 5/0663; A61L 27/3821; A61L 27/3834; A61K 2035/124

(57) ABSTRACT

Methods, systems and activated human mesenchymal stromal cells (hMSCs) from bone marrow regions distal from bone fractures, the hMSCs enhanced for encouraging healing of bone fractures. Systems and methods of activation for enhancing bone fracture healing hMSCs, and the enhanced cells themselves, by exposing and activating distally obtained bone marrow aspirate to acoustic energy prior to placement of the activated cells at a site of a bone fracture.

### CROSS-REFERENCE TO RELATED APPLICATIONS

The application claims the benefit of U.S. Provisional Application No. 62/040,222 filed on Aug. 21, 2014, the entire contents of which are hereby incorporated by reference herein.

### BACKGROUND

This present disclosure relates generally to human mesenchymal stromal cells (hMSCs) isolated from different bone marrow and their use in bone fracture healing.

Musculoskeletal disorders affect the body's muscles, bones, joints, tendons, ligaments and nerves and are the leading cause of chronic disabilities in adults. Significant research efforts have been undertaken during the last decades to ease this disability and improve patients' mobility and quality of life. Bone fracture repairs, pseudarthrosis, and osteochondral defects have been intensively investigated at both the clinical and fundamental level; still, 5-10% of fractures result in either delayed repair (delayed union) or no repair (nonunion). At present there are two primary treatment strategies: (1) surgical intervention that implies the use of bone autograft/allografts, demineralized bone matrix or synthetic materials; and (2) noninvasive treatments such as the application of acoustic energy to the skin of a patient near or over a fracture in a patient (e.g., the Exogen 4000+® Ultrasound Bone Healing System, Smith & Nephew, Inc., Memphis, Tenn.).

Bone regeneration has posed many challenges including, at least because the above described strategies rely on a patient's own cells in situ at the site of the bone fracture healing (BFH)—either stem and/or committed cells—to induce bone regeneration. This may pose challenges at least because in many regions where bone fractures occur, BFH stem and/or committed cells are missing, low in number, and/or are less active than those located in bone marrow of other regions of a patient's bone. To address this, cell-based alternatives, such as the use of human mesenchymal stromal cells (hMSCs) isolated, stimulated (primed) and applied to a BFH site from bone marrow regions distal the site have been studied. As discussed below, priming (or activating) is accomplished by exposure of bone marrow (BM) derived cells to acoustic energy and/or to certain in vitro plating conditions (e.g., but not limited to, plating in serum free media at certain concentrations and isolating adherent cells). See, Leung K S, Lee W S, Tsui H F, Liu P P, Cheung W H, Complex tibial fracture outcomes following treatment with low-intensity pulsed ultrasound, Ultrasound in medicine & biology 2004; 30(3): 389-95.)

Moreover, a reported 42% acceleration in fracture healing in patients exposed to twenty minutes daily ultrasound treatment may not give an excellent therapeutic response. See, Leung et al. above. Mechanical stimulation has further been shown to pre-commit hMSCs towards the osteogenic lineages. (See, e.g., Nikukar H, Reid S, Tsimbouri P M, Riehle M O, Curtis A S, Dalby M J, Osteogenesis of mesenchymal stem cells by nanoscale mechanotransduction, ACS nano 2013; 7(3): 2758-67).)

Cell-based therapies have also been used to expand BM cells where the isolation procedure plays an important role in the selection of desired cell populations. The isolation of hMSCs from BM is mainly achieved by plastic adherence and it is recognized that the number of mononuclear cells (MNCs) plated as well as the culture media have a strong influence on the selection of certain hMSCs populations. (See, e.g., Sacchetti B, Funari A, Michienzi S, et al., Self-renewing osteoprogenitors in bone marrow sinusoids can organize a hematopoietic microenvironment, Cell 2007; 131(2): 324-36).

### SUMMARY

An aspect of the present invention relates to a method of using human mesenchymal stromal cells (hMSCs) from bone marrow regions distal from a bone fracture for encouraging healing of a bone fracture. In aspects of such methods, the aspirated hMSCs may be activated (encouraged in assisting bone fracture healing (BFH)) by exposure to acoustic energy. Once activated, the acoustic energy exposed hMSCs may be placed into a patient's site of a bone fracture to encourage healing.

Further, in embodiments related to those described above, the activation may be performed by an acoustic stimulation device.

Still further, in embodiments of the invention, the acoustic stimulation device may expose the bone marrow aspirate to about 300 Hz of acoustic energy. In yet other embodiments, the acoustic stimulation device may expose the bone marrow aspirate to an acoustic energy standing wave. Further, in yet other embodiments, the acoustic energy of the acoustic energy standing wave may be, but need not be limited to, about 300 Hz.

In additional embodiments, the acoustic stimulating device may comprise a chamber for holding the bone marrow aspirate. Additionally, an acoustic stimulating device may comprise an acoustic energy generator. In some aspects of the invention, the acoustic energy generator may be a speaker.

Other aspects of the invention relate to the creation of an isolated human mesenchymal stromal cell (hMSC) isolated from bone marrow aspirate obtained from a site distal to a bone fracture and activated in vitro for enhanced healing properties for bone fracture healing. In certain embodiments, such cells may be characterized by at least one of the following properties associated with of enhanced bone fracture healing: self-regeneration potential, multilineage potential, proliferation potential, and surface marker expression. In certain embodiments of the invention the activated bone fracture hMSC may be characterized by the cells expressing cell markers bound by at least one antibody of the group CD90, CD73, CD146, CD105, CD271, CD34, CD14, CD79a, HLA-DR, and CD45.

In yet further embodiments of the invention, the isolated hMSC with enhanced healing properties for bone fractures is associated with exposing the aspirated bone marrow to acoustic energy.

Yet further, in some instances, the exposure of the aspirated bone marrow to acoustic energy may be performed in an acoustic stimulation device. In additional aspects, the enhanced bone fracture healing is performed by an acoustic stimulation device that exposes the bone marrow aspirate to about 300 Hz of acoustic energy. Still further, in certain embodiments, the enhanced bone fracture healing is performed by an acoustic stimulation device that exposes the bone marrow aspirate to an acoustic standing wave. Further still, the acoustic standing wave may be at a frequency of about 300 Hz.

Additional embodiments of the invention relate to systems for treating bone fractures by using human mesenchymal stromal cells isolated from regions of bone marrow distal to a bone fracture. Such systems may, for example, include an acoustic energy stimulator for exposing aspirate taken from a bone marrow from a region distal to a bone fracture, the aspirated cells comprising human mesenchymal stromal cells (hMSC). Such systems may also include, for example, an acoustic energy generator as a source of acoustic energy. In certain aspects of the invention, the source of acoustic energy may be a speaker.

In further embodiments, the acoustic energy stimulator may expose the bone marrow aspirate to about 300 Hz. In yet further aspects, the acoustic energy stimulator exposes the bone marrow aspirate to an acoustic standing wave. Yet further, in embodiments of the invention, the acoustic energy stimulator may expose the bone marrow aspirate to an acoustic standing wave at about 300 Hz.

In additional embodiments of the invention, the acoustic energy stimulator may have a tube for draining hMSC after they are exposed to acoustic energy.

### BRIEF DESCRIPTION OF THE DRAWINGS

So that the above recited features and advantages can be understood in detail, a more particular description, may be had by reference to the embodiments thereof that are illustrated in the appended drawings. It is to be noted, however, that the examples illustrated are not to be considered limiting of its scope. The figures are not necessarily to scale and certain features and certain views of the figures may be shown exaggerated in scale or in schematic in the interest of clarity and conciseness.

FIG. 1 is a schematic representation of exemplary experimental designs of exemplary embodiments of the invention.

FIG. 2 is a flowchart of an exemplary embodiment of the invention showing an illustrative method of priming cells for a healing bone fracture.

FIG. 3A is a schematic diagram of an acoustic stimulating device showing the fluid flow within the processing chamber and the introduction of acoustic energy into bone marrow aspirate.

FIG. 3B is a schematic view of a processing chamber showing a source of acoustic energy (e.g., a speaker) located on the bottom of the processing chamber and a standing wave pattern formed in bone marrow aspirate when the acoustic energy of the speaker is provided at 300 Hz.

FIG. 3C is a perspective view of the processing chamber showing the fluid flow within the processing chamber and the formation of a standing wave.

FIG. 3D is a perspective view of the processing chamber showing the speaker located on the bottom of the processing chamber (also shown at the bottom of the processing chambers of FIGS. 3A-3B).

FIG. 3E is a perspective view of the processing chamber showing a standing wave pattern formed in bone marrow aspirate when the acoustic energy of the speaker is provided at 300 Hz.

FIG. 4 is a schematic diagram showing platelets when they are resting, being placed in the processing chamber and treated with acoustic energy, and platelets after they have been activated with acoustic energy in the processing chamber.

FIG. 5 is a chart showing Alizarin red staining of calcium nodules after osteogenic induction of hMSCs isolated under varying culture condition from different donors.

FIGS. 6A-6C are graphs showing characterization of BM aspirated from different locations.

FIGS. 7A1-A4, 7B, 7C1-C4, 7D, 7E1-E4, 7F, 7G1-G4, 7H, 7I1-I4, and 7J show biologic characterization of hMSCs isolated from different BM locations.

FIGS. 8A-8J show biologic characterization of isolated hMSCs from acoustic stimulated BM at 300 and 500 Hz for 5 and 10 minutes.

FIGS. 9A-9F show biologic characterization of hMSCs isolated from BM under different isolation procedures.

FIGS. 10A-10F show biologic characterization of isolated hMSCs from acoustically stimulated BM at 300 Hz for 5 minutes at different volumes.

FIGS. 11A1-A2, 11B, 11C, 11D1-D2, 11E, and 11F show surface marker expression (in percentage) represented as a bar plot; each bar stands as the average over the percentage of surface markers obtained from three donors. Selected sets of cell surface markers expressed positive on hMSCs. All the other investigated sets were expressed negative for both conditions, therefore not shown. No statistical significant differences were found between the two conditions.

### DETAILED DESCRIPTION

In the following description, numerous details are set forth to provide an understanding of the present disclosure. However, it will be understood by those skilled in the art that the present disclosure may be practiced without these details and that numerous variations or modifications from the described embodiments are possible.

### Introduction

The disclosure provides characterized phenotypes, yield, proliferation and multilineage differentiation potential of human mesenchymal stromal cells (hMSCs) isolated from bone marrow (BM) aspirated from different locations in the lower limbs, such as the ilium, proximal femur, distal femur and proximal tibia and examined the inter- and intra-donor variation between the BM-derived hMSCs from these different locations. Bone marrow type, volume, number of mononuclear cells, as well as number of hMSCs and their self-renewal, multilineage potential and surface marker profiling were analyzed, as was the ability of these cells to assist in bone fracture healing.

Additionally, systems and methods of activating BM-derived hMSCs and evaluating their effects on osteogenic/chondrogenic differentiation potential and ability to assist in tissue regeneration upon reimplantation. Two distinct activating strategies were explored: (1) the use of acoustic energy applied on BM in vitro and (2) varying the initial culture conditions of isolated hMSCs in vitro. For example, the use of BM acoustically stimulated ex vivo then injected at a bone fracture site and an evaluation of the impact of the treated BM on bone fracture healing (BFH) have been provided.

Additionally, cell-based therapies may involve the in vitro expansion of cells, where the isolation procedure plays an important role in the selection of desired cell population. (See, e.g., Sacchetti B, Funari A, Michienzi S, et al. Self-renewing osteoprogenitors in bone marrow sinusoids can organize a hematopoietic microenvironment, Cell 2007; 131(2): 324-36). The isolation of hMSCs from BM may be mainly achieved by plastic adherence and the number of mononuclear cells (MNCs) plated as well as the culture media may have a strong influence on the selection of certain hMSC populations. Accordingly, untested low hMSC seeding density and/or serum free media (SF) might select hMSCs prone to higher self-renewal potential. For example, whether the use of SF media might select hMSCs prone to chondrogenic differentiation potential is examined. The phenotype of the isolated hMSCs under previously discussed procedures for isolation are compared, and the isolation of hMSCs with enhanced potential to assist in BFH is generated.

Still further, optimal ratios between aspirated BM volume and hMSC concentration is examined to explain the differences in femur/tibia fracture healing and to propose new methods to accelerate BFH.

### Materials and Methods

### Aspiration of Bone Marrow

BM aspirates were obtained from patients undergoing total hip arthroplasty (THA) or total knee arthroplasty (TKA). An 8G Jamshidi BM needle fit with a 50-mL Luer lock syringe containing 1 mL of 1,000 U heparin per 10 mL of BM was used to aspirate the BM. Subsequently, the BM was transferred to blood collection tubes (BD-367526) for the transport from the operating theatre to the laboratory. The BM was kept at ambient room temperature until being processed within the same day.

BM was aspirated from 4 different locations: the supra acetabular sulcus (ilium) in twelve donors, the medullary cavity or lateral diaphysis of the femur (proximal femur) in seven donors, and the epiphysis or medullary cavity from the distal femur or proximal tibia in seven donors.

The collection and anonymous use of the BM aspirate was performed in compliance with the relevant laws and institutional guidelines of the Medisch Spectrum Twente, Twente Ethische Toetsings Commissie. Patients provided fully informed consent after being provided with a verbal explanation and an opportunity for questioning.

Isolation and Culture of hMSCs

BM aspirate was passed through a 70 μm pore-size cell strainer to remove the presence of tissue pieces, after which MNC concentration was analyzed using the Beckman coulter ACT diff 2. Due to device limitations only BM aspirate from the ilium and proximal femur could be analyzed and not from the distal femur and proximal tibia. Based on the isolation method used, different concentrations of MNCs/cm² were plated. We defined three isolation/culture conditions: heterogeneous, multiclonal and SF plating condition.

For the heterogeneous isolation condition, BM aspirate was plated at a density of 5×10⁵ MNCs/cm² and cultured in growth media (GM) consisting of α-minimal essential media (αMEM, LIFE TECHNOLOGIES®), 10% Fetal Bovine Serum (FBS, GIBCO®), 0.2 mM L-ascorbic acid 2-phosphate magnesium salt (SIGMA®), 2 mM L-glutamine (GIBCO®), 100 units/mL penicillin (Gibco) and 100 mg/mL streptomycin (GIBCO®).

For the multiclonal isolation condition, BM aspirate was plated at a clonal density of 5×10⁴ MNCs/cm² and cultured in GM.

For the SF condition, BM aspirate was plated at a cell density of 1.5×10⁶ MNC/cm² in α-MEM containing no additives for the first three days.

At day four, the non-adherent cell fraction was removed and the media was changed to GM for all three conditions. Hereafter, media was refreshed twice a week. At semi-confluence cells were trypsinized and used for sub-culturing or stored in liquid nitrogen for future use.

In total, BM was aspirated from 19 donors and subsequently divided between the different experiments. BM from 14 donors was used to evaluate the most convenient aspiration site location. BM was plated under heterogeneous condition, with exception of distal Femur and proximal Tibia where 2 ml BM was plated each time, as the initial amounts of MNCs were unknown. BM from 11 donors was used to evaluate the effects of acoustic energy. The BM was plated under heterogeneous condition. And BM from 6 donors was used to evaluate the effects of varying the initial isolation conditions of the hMSCs.

Donor information, BM aspiration location, BM volume and concentration of MNCs/ml can be found in Table 1 below. BM was cultured at 37° C. and 5% CO².

TABLE I
Donor and BM information. From left to right: donor number/gender/age, BM aspiration
location, type, aspiration location and concentration of MNCs/ml.

| | | | | | BM | |
|---|---|---|---|---|---|---|
| | | | | | VOLUME | MNC/ML |
| DONOR | GENDER | AGE | BM LOCATION | COLOR | (ML) | (*E+06) |
| Donor 1 | ♂ | 61 | Ilium | Red | 20 | 31.6 |
| Donor 2 | ♀ | 50 | Ilium | Red | 35 | 12.3 |
| Donor 3 | ♀ | 71 | Ilium | Red | 15 | 17.9 |
| Donor 4 | ♂ | 64 | Ilium | Red | 25 | 20.2 |
| Donor 5 | ♂ | 79 | Ilium | Red | 30 | 13.5 |
| Donor 6 | ♀ | 76 | Ilium | Red | 28 | 6.8 |
| | | | Proximal Femur | Red | 11.5 | 16.3 |
| Donor 7 | ♀ | 68 | Ilium | Red | 20 | 16.1 |
| | | | Proximal Femur | Red | 1 | 15.1 |
| Donor 8 | ♀ | 64 | Ilium | Red | 13 | 34.7 |
| | | | Proximal Femur | Red | 6 | 16.2 |
| Donor 9 | ♂ | 61 | Ilium | Red | 20 | 7.3 |
| | | | Proximal Femur | Red | 6.5 | 7.9 |
| Donor 10 | ♀ | 68 | Ilium | Red | 22.5 | 14.5 |
| | | | Proximal Femur | Red | 9 | 22.4 |
| Donor 11 | ♀ | 76 | Ilium | Red | 22.5 | 7.4 |
| | | | Proximal Femur | Red | 5 | 6.4 |
| Donor 12 | ♀ | 67 | Ilium | Red | 7.5 | 26.6 |
| | | | Proximal Femur | Red | 5 | 37 |
| Donor 13 | ♂ | 75 | Distal Femur | Yellow | 3.5 | — |
| | | | Proximal Tibia | Yellow | 2.5 | — |
| Donor 14 | ♂ | 65 | Distal Femur | Yellow | 5 | — |
| | | | Proximal Tibia | Yellow | 7.5 | — |
| Donor 15 | ♂ | 60 | Distal Femur | Yellow | 9 | — |
| | | | Proximal Tibia | Yellow | 3 | — |
| Donor 16 | ♀ | 65 | Distal Femur | Yellow | 2 | — |
| | | | Proximal Tibia | Yellow | 1.5 | — |
| Donor 17 | ♂ | 66 | Distal Femur | Yellow | 6 | — |
| | | | Proximal Tibia | Yellow | 7 | — |
| Donor 18 | ♀ | 54 | Distal Femur | Yellow | 8 | — |
| | | | Proximal Tibia | Yellow | 4.5 | — |
| Donor 19 | ♀ | 63 | Distal Femur | Yellow | 2 | — |
| | | | Proximal Tibia | Yellow | 2 | — |

hMSC Population Doubling

To assess hMSC proliferation, cells from passage one were seeded in GM at 5,000 cells/cm² in T175 tissue culture flasks. At semi-confluence the cells were trypsinized and counted. Population doubling (PD) was calculated according to the formula PD=log₂(N_{E}/Nᵢ), where N_{E} and N_{I} are the number of hMSCs obtained at passage 2 (P2) and passage 1 (P1), respectively.

### Colony Forming Unit and Colony Forming Unit-Osteoblast Potential (Mineralization)

A colony forming unit (CFU) assay was used as an indicator of self-renewal potential of the hMSCs and a CFU-osteoblast (CFU-Ob) assay was used as an indicator of osteoprogenitor presence in the formed CFUs. Two million MNCs were seeded in duplicate in T25 culture flasks and grown in GM for the first 7 days, followed by transition to mineralization media for further 7 more days. Mineralization media consisted of GM containing 0.01 M β-glycerophosphate (BGP) and 10⁻⁸ M Dexamethasone (Dex), all from SIGMA®. At day 14, cultures were fixed with 10% formalin for 15 min at ambient temperature, after which alkaline phosphatase (ALP) positive colonies were stained using the Leukocyte Alkaline Phosphatase Kit-ALP (SIGMA®) following manufacturer's instructions. Subsequently, the total amount of colonies formed were stained using 0.5% Coomassie Blue staining solution added to the monolayer for 10 minutes. Images of the stained colonies were acquired using an Epson Perfection V750 PRO scanner. Total number of CFUs and ALP positive colonies was quantified using ImageJ 1.45s software and the percentage of ALP positive CFUs calculated.

### Alizarin Red Staining (Mineralization)

hMSCs were seeded in triplicate at 50,000 cells/well in T25 and grown in control media consisting of GM containing 0.01 M BGP and in mineralization medium consisting of GM containing BGP and 10⁻⁸ M Dex. The media was refreshed twice a week. After 28 days, cells were fixed in 10% formalin for 15 minutes at ambient temperature and stained with 2% Alizarin red solution (SIGMA®) for 5 minutes. Images were captured using a NIKON® bright field microscope.

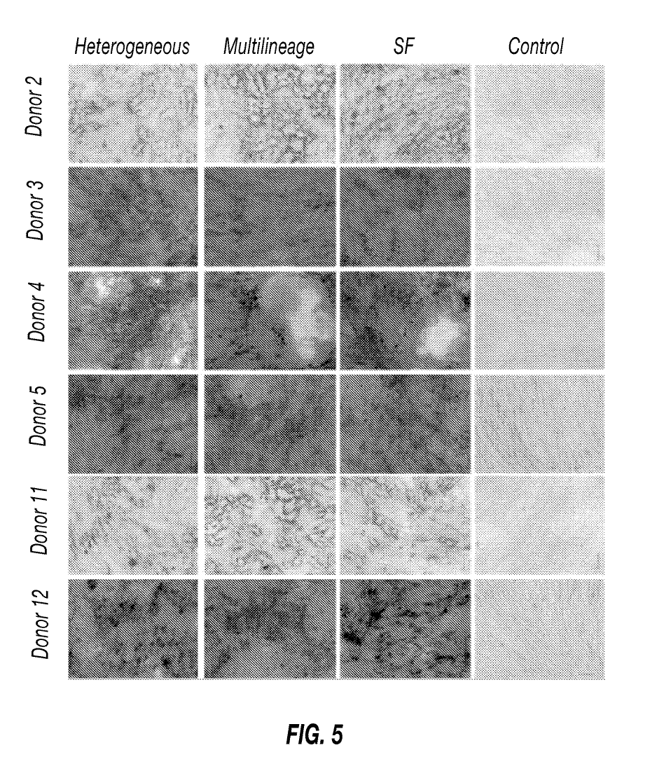

FIG. 5 shows Alizarin red staining of calcium nodules after osteogenic induction of hMSC isolated under varying culture condition from different donors. No differences were observed between the culture conditions, though differences between the donors were identified, donor 2 and 11 showed less calcium nodules formation than the rest of the donors. All the controls stained negative for calcium nodules formation. Values are represented as mean±standard deviation of at least three independent experiments (n=3).

### Nodule Size Formation (Chondrogenesis)

hMSCs were seeded in quadruplicate at 100,000 cells/well in a 384-well plate in chondrogenic control media (CCM) consisting of GM (without serum), 50 μg/mL insulin transferrin selenium-premix (SIGMA®) and 40 μg/mL proline (SIGMA®) and incubated for 24 h to allow cell-adhesion. The next day the medium was refreshed in the chondrogenic control wells and changed to chondrogenic differentiation medium, consisting of CCM containing 10 ng/mL transforming growth factor beta 3 (R&D SYSTEMS®) and 10⁻⁷ M Dex, in the other wells. After seven days the formed nodules were fixed in 10% formalin for 15 min, at ambient temperature and images were captured using a NIKON® bright field microscope. The nodule area and the number of nodules formed were quantified using ImageJ® 1.45s software. The early cell condensation phenotype was associated with induction of chondrogenesis, previously described by Johnson K et. al. (Johnson K, Zhu S, Tremblay M S, et al., A stem cell-based approach to cartilage repair, Science 2012; 336(6082): 717-21.)

### Oil Red O Staining (Adipogenesis)

hMSCs were seeded in triplicate at 25,000 cells/well in 24-well plates and grown in control medium consisting of GM or adipogenic medium consisting of GM containing 0.2 mM indomethacin, 0.5 mM isobutylmethylxanthine, 10⁻⁶ M Dex and 10 μg/mL human insulin, all from SIGMA®. The media was refreshed twice a week. After three weeks the cells were fixed with 10% formalin for 15 minutes at ambient temperature, following which the cell monolayer was incubated for 5 minutes in 60% isopropanol, followed by staining with Oil red O solution (3 mg/mL in 60% isopropanol). After five minutes, samples were rinsed with demineralized water and images were captured using a Nikon® bright field microscope. Following imaging, Oil red O staining was extracted from the cells in 4% Igepal in isopropanol and absorbance was measured at 540 nm (Lambda 40; PERKIN ELMER®). One hundred percent Oil red O was included in the calibration curve measurements, from which the percentage of Oil red O staining was calculated.

### Flow Cytometry

hMSCs at passage three or four were expanded in T175 until they reached confluence. Cells were trypsinized and incubated for 30 minutes in blocking buffer consisting of 17% bovine serum albumin (SIGMA®) in PBS, followed by incubation with FITC- or PE-conjugated mouse anti-human antibodies for 30 minutes at 4° C. in the dark. Samples were then washed three times with a washing buffer consisting of 3% bovine serum albumin in PBS. Expression levels were analyzed using FACSARIA® flow cytometer (BD BIOSCIENCE®). For phenotypic characterization the following antibodies were used: CD90, CD73, CD146, CD105, CD271, CD34, CD14, CD79a, HLA-DR, CD45 and IgG1 and Ig G2a as isotype controls (all from BD PHARMING®).

### Acoustic Stimulation of Bone Marrow

Acoustic stimulation of BM was achieved using the bone marrow aspirate concentration device, previous described by Ridgway et. al. B M was placed into the processing chamber of the device and acoustic vibration was applied using a voice-coil which produced a geometric standing waveform pattern on the BM fluid surface. Different frequencies were tested by manual adjustment using an Oscilloscope (Agilent Technologies, InfiniVision, MSO-X-3014A Mixed Signal Oscilloscope) and two frequencies (300 and 500 Hz) were selected for further experimental research. The BM was processed for 5 and 10 minutes for both selected frequencies. Baseline was defined as unstimulated BM.

### Bone Marrow Viscosity

BM viscosity from 6 donors (3 donors for ilium and proximal femur and 3 donors for distal femur and proximal tibia) was measured using the Rheometer Physica MCR-301®. A total of thirty different points, with increasing shear rate from 0 to 250 l/s and periodic pause of 10 seconds between each point, were measured. The volume of BM used for the measurements was 350 μL per measuring cycle. All samples were measured in duplicates at ambient room temperature.

### Statistical Analysis

Statistical analysis was performed using GRAPHPAD PRISM 6® software. Two-way ANOVA and a Bonferroni post-test or Tukey was used to compare the different conditions. Uniform distribution of data, to test inter-donor variation, was assessed using a Chi-squared test. A P≦0.05 indicates a statistically significant difference. Results are shown as mean±standard deviation.

### Experimental Results

### Exemplary Devices and Methods

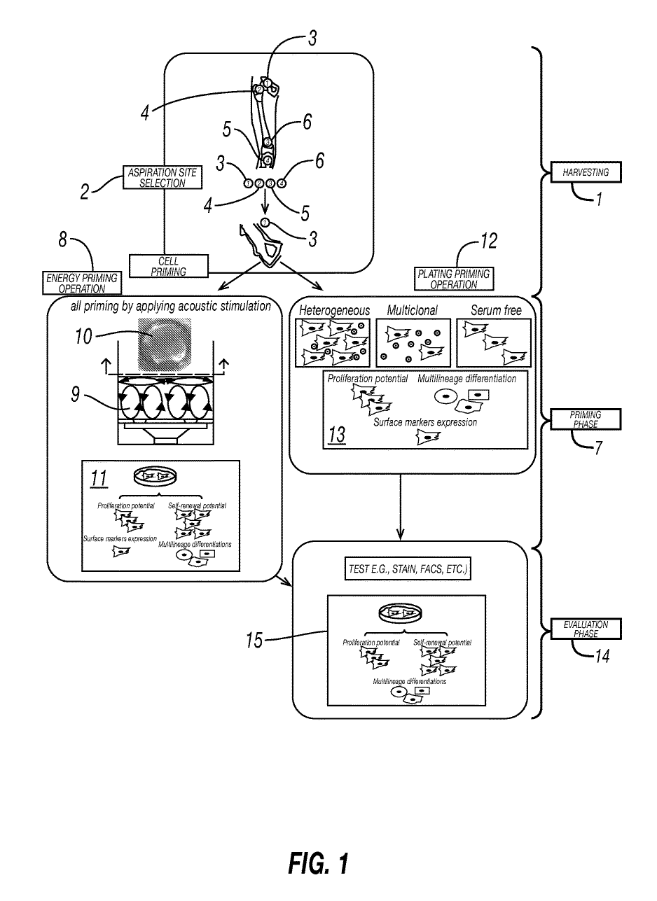

FIG. 1 is a schematic representation of an experimental design of exemplary embodiments of the invention. In a first phase, a Harvesting Phase 1, bone marrow is harvested (in an Aspiration Site Selection Step 2) from four (or more or less) locations 3-6 distal to the BFH site in the same or a separate bone(s).

FIG. 1 also shows a Priming Phase 7, where bone marrow cells isolated from the various bone regions in the Harvesting Phase 1 are primed to increase their bone fracture healing (BFH) capabilities when reapplied to a patient's bone fracture in vivo. As discussed and shown in more detail below, in embodiments of the invention, isolated BM cells may be primed by one of two (or both of two) methods. The left box, Energy Priming Operation 8, schematically shows an acoustic stimulation device 9 (described in more detail below). Top view 10 is a schematic view of device 9 looking downward on the BM while it is being exposed to acoustic energy. The application of acoustic energy to BM aspirate can enhance the bone fracture healing capabilities of the stimulated (treated) hMSC when placed in vivo at a BFH site (as discussed below). The lower half of the left box in FIG. 1 (Energy Priming 11) shows embodiments wherein acoustic energy primed cells may be tested in vivo for various properties associated with increased BFH as discussed in detail herein. These include but are not limited to testing for self-regeneration potential, proliferation potential, and surface marker expression.

In embodiments of the invention, these testing steps may be omitted and the cells once activated (primed) with acoustic energy applied directly to a patient's bone fracture to enhance BFH.

FIG. 1 also shows a Priming Phase (Plating Operation) 12 showing an alternative embodiment (mentioned above and discussed below) wherein BM cells are isolated and plated (primed) by in vivo plating of cells in varying cell culture conditions, including under heterogeneous, musoconal, and serum free (SF) media. Similar to the energy priming 11, the resulting cells were then tested 13 for various phenotypes associated with increased BFH including self-regeneration potential, multilinage potential, proliferation potential, and surface marker expression.

FIG. 1 further shows an Evaluation Phase 14 including a box labeled Test (e.g., stain, FACS, ETC.) 15. This box shows additional tests that may be performed on the primed cells as discussed above to assess their capability for BFH. All of the isolated cells from the acoustically primed and/or platted primed cells may be subject to these additional tests (in sterile conditions, as are all previous steps) and the most promising cells isolated therefrom and implanted. Alternatively, these tests may be performed on samples from batches of primed cells to assure that the entire sample shows preferred BFH properties, yet is not subjected to additional testing.

As mentioned above, these testing steps are not necessarily required and may be shown here for the sake of completeness of how the invention was initially discovered and characterized. For example, in embodiments of the invention, these and/or all testing steps may be omitted and the cells once activated (primed) with acoustic energy applied directly to a patient's bone fracture to enhance BFH. The same may be done with the cell platting activated cells.

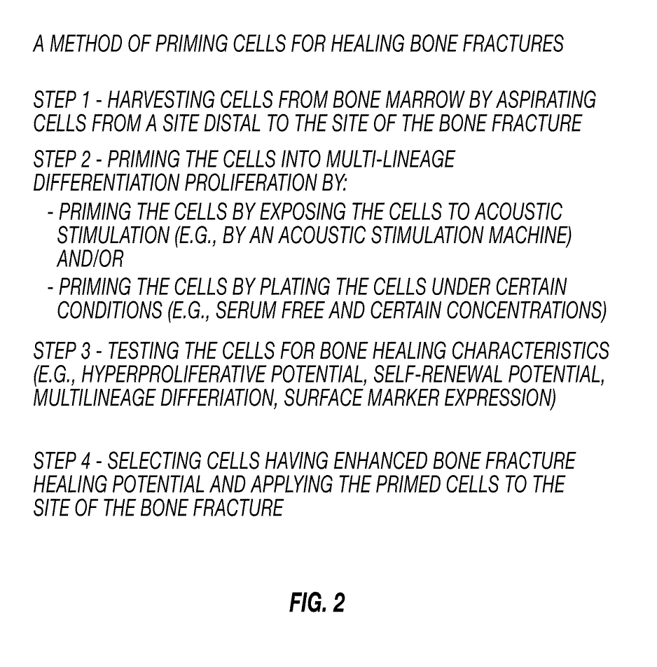

FIG. 2 is a flow chart showing certain embodiments of methods of the invention. In a step 1, BM cells are harvested by aspiration from BM at selected sites distal from the bone fracture to be healed. In a step 2, aspirated BM cells are primed, in vitro, to enhance their ability for BFH. The priming process may involve alternatively, or in any sequence, exposing the cells to acoustic energy (e.g., in an acoustic stimulation device, discussed later). In other embodiments, the cells may be primed by a process of plating the cells under conditions to enhance their BFH abilities in vivo (described later, for example in SF medium at low concentration). The primed hMSC may be applied to a bone fracture site in vivo.

Step 3 involves testing the cells for bone healing characteristics. Such testing may involve, for example, hyperproliferative potential, self-renewal potential, multilineage differentiation, and/or surface marker expression. Step 4 involves selecting cells having enhanced bone fracture healing potential and applying the primed cells to the site of the bone fracture.

Steps 3 and 4, as discussed above, may be eliminated when performing the invention. For example, in embodiments of the invention, once cells have been harvested from bone marrow sites distal to a bone fracture and subsequently exposed to acoustic energy, the cells may be next applied directly to a bone fracture (i.e., with no intervening testing and/or selecting steps). The same is true for embodiments of platting priming of cells, wherein in certain embodiments the cells may be removed from plates and applied directed to bone fractures, with no intervening selecting and/or testing steps. In aspects of the invention, the testing and selecting steps shown in FIG. 2 may relate solely to the initial characterization and proof of function of the instant inventions.

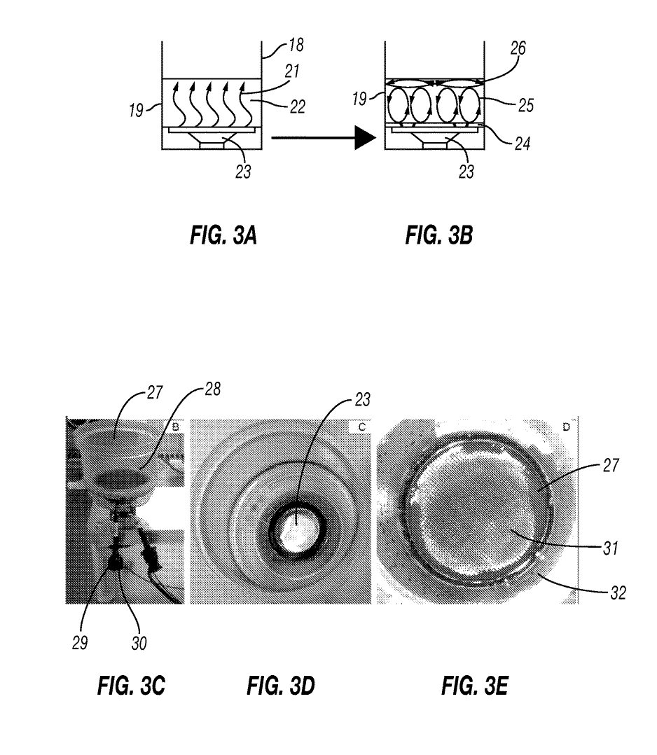

FIG. 3A is a schematic side view of an exemplary embodiment of an acoustic stimulating device 18 showing the acoustic energy sound waves 21 and bone marrow aspirate 22 within the processing chamber 19. A source of acoustic energy (e.g., a speaker) 23 is shown located at the bottom of the processing chamber 19. In this embodiment, a relatively small amount, 4 mL, of BM aspirate was exposed to acoustic stimulation from acoustic source 23 at 300 Hz. Exemplary embodiments of flow chambers shown herein may include, but are not limited to, those disclosed in U.S. Pat. No. 8,273,253, the entire contents of which is hereby incorporated by reference herein.

FIG. 3B is a schematic side view of the processing chamber 19, and is similar thereto, only showing at least in addition an acoustic standing wave 25 (instead of streaming acoustic energy), an oscillating free surface 26, and a viscous boundary layer 24 located at the bottom of the processing chamber 19. The acoustic standing wave and other features in FIG. 3B are generated by an acoustic generating device 23 (e.g., but not limited to a speaker) emitting acoustic energy at 300 Hz.

FIG. 3C is a perspective view an acoustic stimulating device 27 showing an embodiment of the fluid flow within the processing chamber 28 and the formation of a standing wave (see FIG. 3E, 31). Tubing for flowing bone marrow aspirate into and out of the chamber 28 is shown as 29 and 30, respectively.

FIG. 3D is a perspective view of the processing chamber showing the acoustic stimulator 23 located on the bottom of the device 27 (embodiments of acoustic stimulators 23 are also shown at the bottom of the processing chambers of FIGS. 3A-3B).

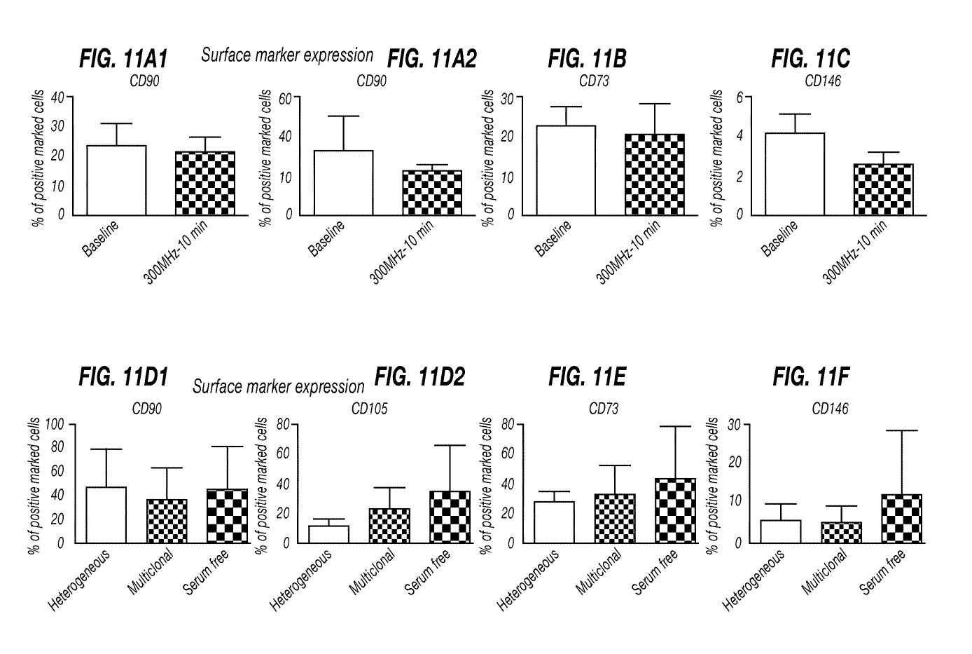

FIG. 3E is a perspective view of the acoustic stimulating (enhancing) device 27 showing a standing wave pattern 31 formed in bone marrow aspirate 32 when acoustic energy of an acoustic stimulator (not shown) is provided at 300 Hz. Refer to FIG. 11, for example, for further information on the properties of cells treated with the device(s) and methods of FIGS. 3A-3E.

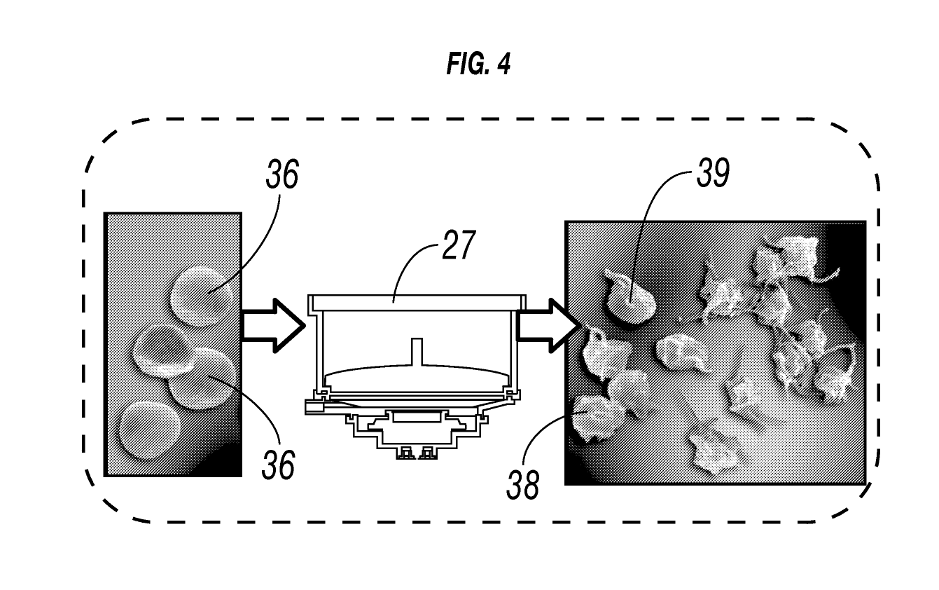

FIG. 4 shows an operation using the device(s) 27 of FIGS. 3A-3D. For example, it shows the effects of an embodiment on platelets 36 when they are resting, before being placed in the processing chamber of device 27 and treated with acoustic energy as described, for example, above in FIGS. 3A-3D.

FIG. 4 also shows platelets 38 (originally platelets 36 of FIG. 4A, before acoustic treatment) after they have been exposed (primed, activated, enhanced) to acoustic energy in the processing chamber 27. Details of the differences of cells before 36 and after 38 treatments in the processing chamber 27 are shown and discussed below.

### Inter-Donor Variability in Bone Marrow Aspirate

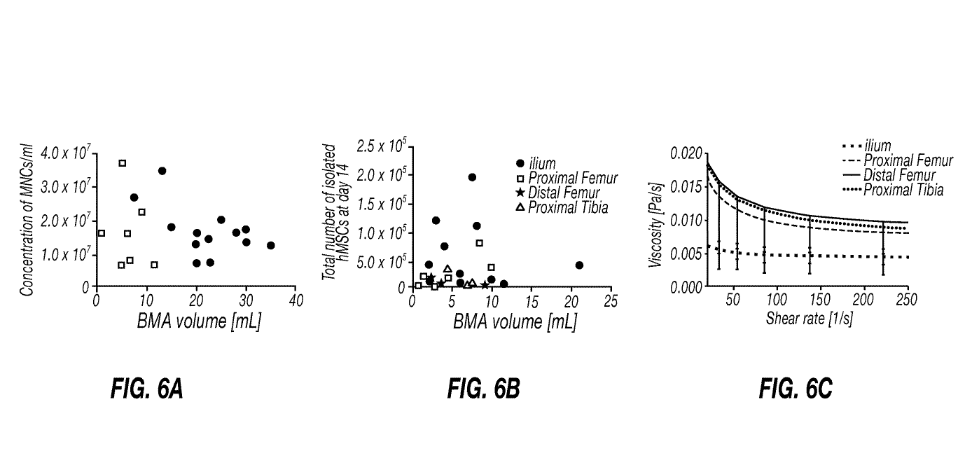

FIGS. 6A-C are graphs showing characterizations of BM aspirated from different locations. FIG. 6A shows correlation between aspirated BM volume and MNCconcentration, for the ilium and proximal femur. FIG. 6B shows correlation between the plated BM volumes and the number of isolated hMSCs, for the heterogeneous isolation condition only. FIG. 6C shows BM viscosity curves from different aspiration locations, represented as correlation between the shear rate and the viscosity. The values represented the mean±standard deviation of three BM donors (n=3). Statistical significant differences were found with ***p<0.001 and **p<0.01.

The age of the donors varied from 50 to 79 years, with an average of 66 years. The volume of BM obtained from the different locations varied significantly with an average of 22±7.6 mL for the ilium, 6±3.3 mL for the proximal femur, 5±2.9 mL for the distal femur and 4±2.6 mL for the proximal tibia. BM volumes from the ilium yielded higher concentration of MNCs for volumes close to 10 mL (2.6×10⁷ MNC/mL), while volumes close and larger than 20 ml, yielded lower concentration of MNCs (1.4×10⁷ MNC/mL). Similarly, BM aspirated from proximal femur showed higher MNC yield for volumes lower than 5 mL, 1.9×10⁷ MNC/mL versus 1.3×10⁷ MNC/mL (FIG. 6A and Table 1).

The concentration of hMSCs obtained at the end of the expansion phase (14 days after MNC seeding) was in average 566,494 hMSC/mL for the ilium, 245,549 hMSC/mL for the proximal femur, 76,250 hMSC/mL for the distal femur and 122,321 hMSC/mL for the proximal tibia samples (FIG. 6B).

Macroscopically, the BM aspirated from the ilium and proximal femur was red, while BM aspirated from distal femur and proximal tibia was yellow, consistent with a higher presence of lipid droplets in the later (image not shown). Compared with the other aspiration locations we observed a significant decrease in BM viscosity for BM aspirated from the proximal tibia (FIG. 6C). Morphological appearance of expanded hMSCs did not show any visible differences between the different BM aspiration locations (data not shown).

Effect of Aspiration Location on Biological Characteristics of hMSCs

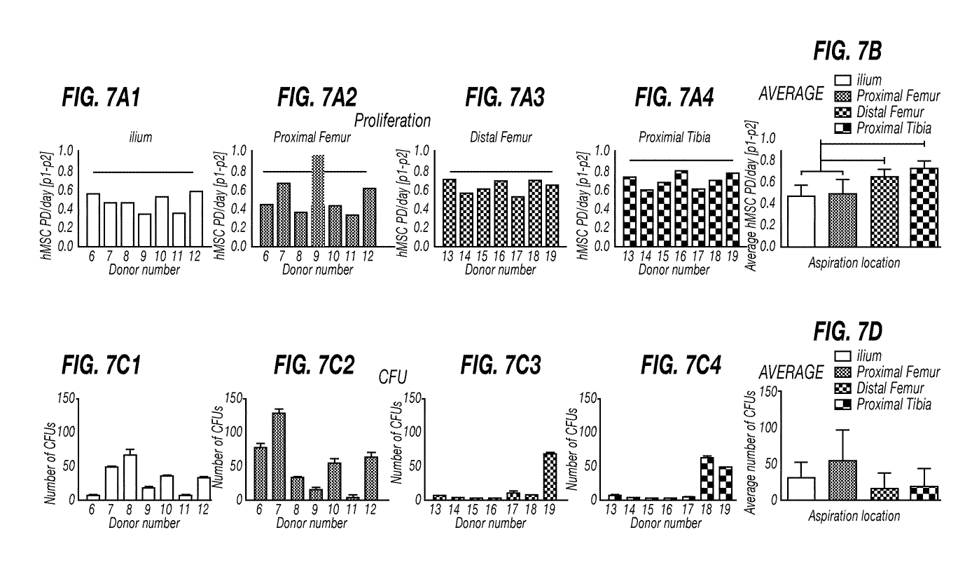

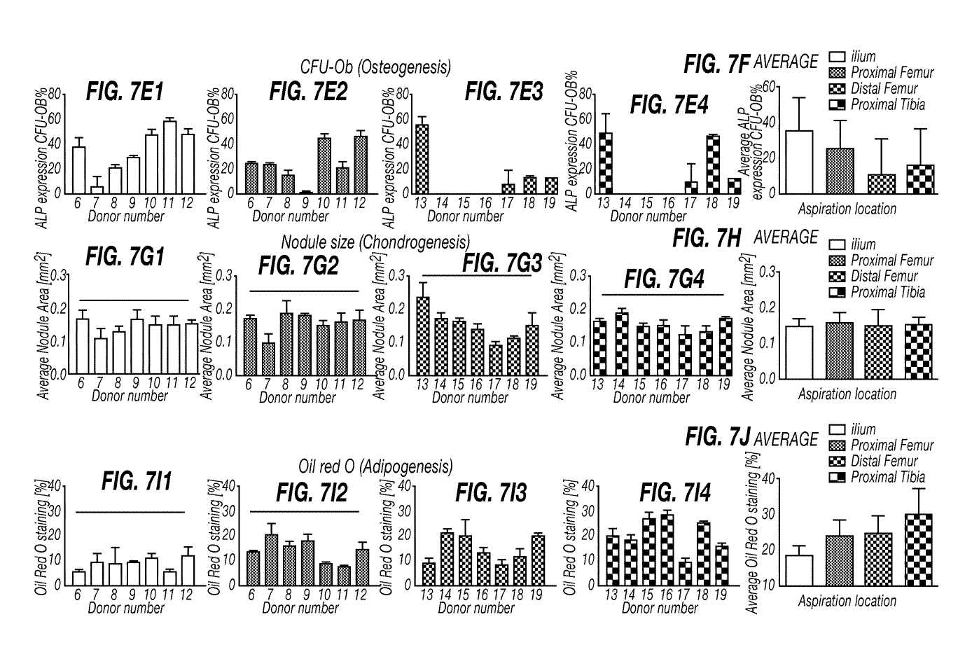

FIGS. 7A1-A4, 7B, 7C1-C4, 7D, 7E1-E4, 7F 7G1-G4, 7H, 7I1-I4, and 7J are graphs showing biologic characterization of hMSCs isolated from different BM locations. FIGS. 7A1-A4 show proliferation of hMSC calculated as PD/day from passage 1 to 2, donor and location dependent. FIG. 7B show Proliferation average over the donors. FIG. 7C1-C4 shows CFU potential of hMSC, donor and location dependent. FIG. 7D shows the CFU average over the donors. FIGS. 7E1-E4 shows Osteogenic potential calculated as percentage of ALP positive colonies within the CFUs, donor and location dependent. FIG. 7F shows Osteogenesis average. FIG. 7G1-G4 shows Chondrogenic potential, quantification of nodule size area in mm², an early cell condensation phenotype associated with induction of chondrogenesis, donor and location dependent. FIG. 7H shows Chondrogenesis average. FIG. 7I1-I4 shows Adipogenic potential, quantification of Oil red O staining relative to 100% Oil red O staining solution, donor and location dependent. FIG. 7J shows Adipogenesis average. The uniform distribution of data, to test inter-donor variation, was assessed using a Chi-squared test and presented as a bar above all donors. Values are represented as mean±standard deviation of at least three independent experiments (n≧3). Statistically significant differences were found with ***p<0.001, **p<0.01 and *p<0.05.

Proliferation, self-renewal and multilineage potential (osteo-, chondro- and adipogenic) were assessed for hMSCs isolated from the different locations (FIGS. 7A1-7J). Proliferation capacity of hMSC showed a uniform distribution over all donors, for each BM aspiration location independently (FIGS. 7A1-A4). An average over all donors showed a statistically significant increase in the proliferation of hMSCs isolated from distal femur (0.64±0.07) and proximal tibia (0.71±0.08) when compared to the ilium (0.47±0.09) and proximal femur (0.48±0.13) (FIG. 7B).

In contrast, CFU capacity of hMSCs showed a non-uniform distribution over all donors, independent of the BM location (FIGS. 7C1-C4). An average of the overall donors shows a trend towards higher CFU capacity of hMSCs isolated from the ilium 31±22 CFU and proximal femur 54±42 CFU than distal femur 14±24 CFU and proximal tibia 19±5 CFU (FIG. 7D).

Similarly, mineralization capacity showed a similar trend with higher CFU-Ob potential in hMSCs isolated from the ilium 12±11 CFU-Ob (36%±18 CFU-Ob/CFU) and proximal femur 11±10 CFU-Ob (26%±16 CFU-Ob/CFU) than distal femur 1±1 CFU-Ob (13%±20 CFU-Ob/CFU) and proximal tibia 5±11 CFU-Ob (17%±22 CFU-Ob/CFU) (FIG. 7F). The high standard deviation is attributed to the non-uniform distribution over the donors (FIG. 7E1-E4).

Chondrogenic potential of hMSCs, determined by the nodule size formation, showed a uniform distribution over all donors, for each BM aspiration location independently (FIG. 7G1-G4). No statistically significant difference was observed between the different BM aspiration locations (FIG. 7H).

Adipogenic potential of hMSCs showed a uniform distribution over all donors for BM aspirated from the ilium and proximal femur but not from distal femur and proximal tibia (FIG. 7I1-I4). An average over all donors showed a significant increase in stained fat droplets in the proximal tibia 21%±6.85 when compared to the ilium 9%±2.5. No statistical significant differences were observed between the other groups (FIG. 7J).

Effect of Acoustic Stimulation on hMSCs

Self-renewal, proliferation and multilineage potential (osteo-, chondro- and adipogenic) were assessed for the acoustic stimulated hMSCs.

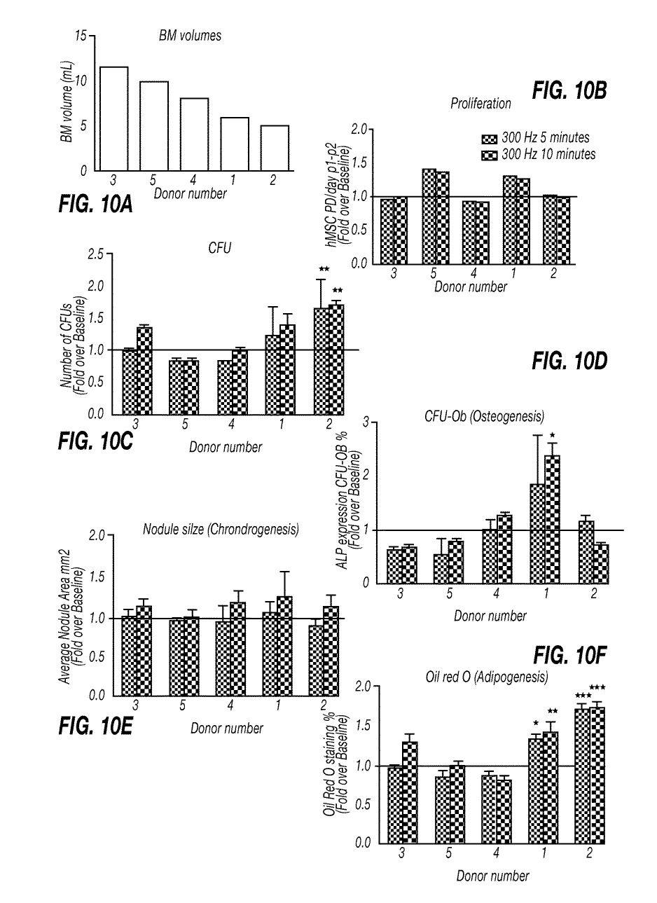

Different BM volumes (11.5, 10, 8, 6, and 5 mL) were stimulated at a frequency of 300 Hz for 5 and 10 minutes. Upon acoustic stimulation a significant increase in CFU, mineralization and adipogenesis was observed for hMSCs isolated from small BM volumes (5 and 6 mL) rather than larger volumes (8, 10 or 11.5 mL). No statistically significant differences were observed in proliferation or chondrogenesis between the conditions (FIGS. 10A-10F). Based on the above-mentioned results subsequent experiments were performed using small BM volumes (4 mL). An illustration of the device, while 4 mL of BM is acoustic stimulated at 300 Hz, is presented in FIGS. 3A-3E.

FIGS. 10A-10F are graphs which show the biologic characterization of isolated hMSC from acoustically stimulated BM at 300 Hz for 5 minutes at different volumes, 11.5, 10, 8, 6 and 5 mL. The results are presented as the fold change over the non-stimulated bone marrow (baseline). FIG. 10A is a graphic representation of the bone marrow volumes, donor dependent. FIG. 10B is a Proliferation of hMSC calculated as PD/day from passage 1 to 2, donor and volume dependent. FIG. 10C is a CFU potential of hMSC, donor and volume dependent. FIG. 10D is a Osteogenic potential calculated as percentage of ALP positive colonies within the CFUs, donor and volume dependent. FIG. 10E is a Chondrogenic potential, quantification of nodule size area in mm², donor and volume dependent. FIG. 10F is a Adipogenic potential, quantification of Oil red O staining relative to 100% Oil red O staining solution, donor and volume dependent. Values are represented as mean±standard deviation of at least three independent experiments (n≧3). Statistically significant differences were found with ***p<0.001, **p<0.01 and *p<0.05.

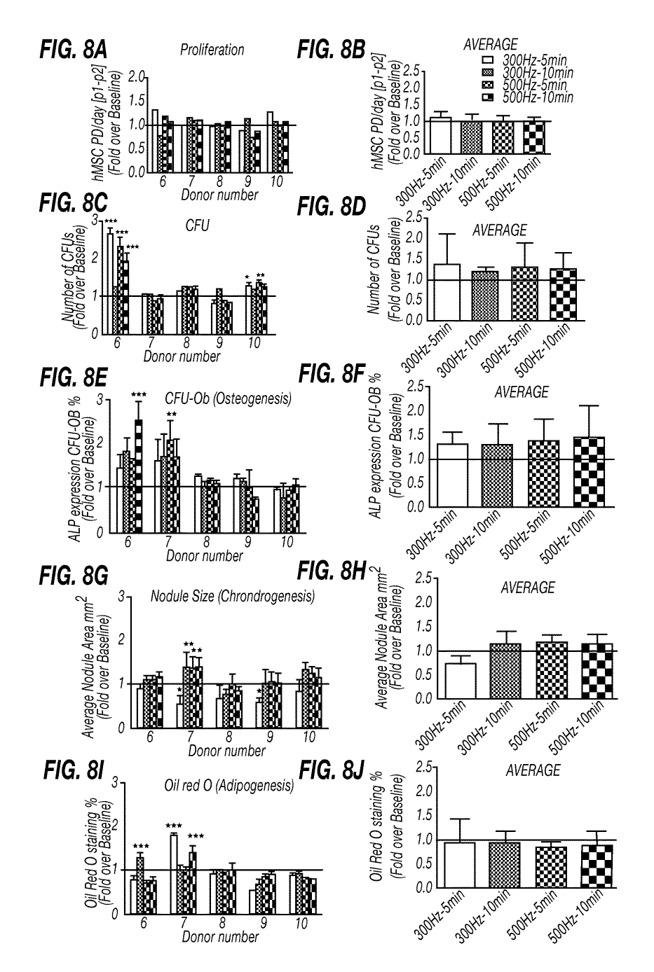

FIGS. 8A-8J are graphs show the biologic characterization of isolated hMSC from acoustic stimulated BM at 300 and 500 Hz for 5 and 10 minutes. The results are presented as the fold change over the non-stimulated BM (baseline). FIG. 8A shows proliferation of hMSC calculated as PD/day from passage 1 to 2, donor and stimulation dependent. FIG. 8B shows the Proliferation average. FIG. 8C shows CFU potential of hMSC, donor and stimulation dependent. FIG. 8D shows CFU average. FIG. 8E shows Osteogenic potential calculated as percentage of ALP positive colonies within the CFUs, donor and stimulation dependent. FIG. 8F shows Osteogenesis average. FIG. 8G shows Chondrogenic potential, quantification of nodule size area in mm², donor and stimulation dependent. FIG. 8H shows Chondrogenesis average. FIG. 8I shows Adipogenic potential, quantification of Oil red O staining relative to 100% Oil red O staining solution, donor and stimulation dependent. FIG. 8J shows Adipogenesis average. Values are represented as mean±standard deviation of at least three independent experiments (n≧3). Statistically significant differences were found with ***p<0.001, **p<0.01 and *p<0.05.

Acoustic stimulation of BM at 300 and 500 Hz for 5 and 10 minutes showed no statistically significant difference in hMSC proliferation between the conditions (FIG. 8A, B). In contrast, a positive trend in CFU, mineralization and chondrogenic, but not adipogenic potential, was observed upon acoustic stimulation (FIGS. 8C-8J).

Surface marker expression on hMSCs isolated from acoustic stimulated BM (300 Hz for 5 minutes) showed a trend towards a decrease in expression of positive surface markers such as CD105, CD90, CD146 and CD73, when compared to the baseline (FIG. 11).

Effect of Varying the Initial Culture Condition on hMSCs

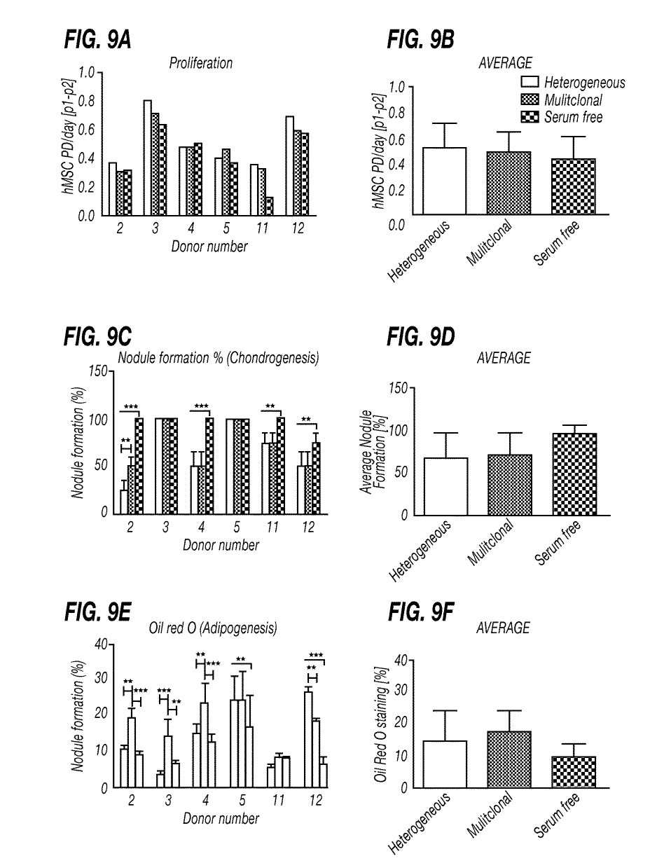

FIGS. 9A-9F are graphs showing results of the isolation of hMSCs from the BM assessed by varying the initial culture conditions, and their proliferation, multilineage differentiation potential, and cell surface marker expression were analyzed. No difference in proliferation (FIGS. 9A-9B) and osteogenesis (FIGS. 7A1-J) was observed between the different isolation conditions. In contrast, isolation of hMSCs under SF condition showed a trend in increased chondrogenesis, with 4 out of 6 donors showing statistically significant increase (FIGS. 9C and 9D), and decreased adipogenesis (FIG. 9F). Isolation of hMSCs under multiclonal condition showed a trend in increased adipogenesis (FIG. 9F). Statistically significant increases in adipogenesis were observed in 5 out of 6 donors in multiclonal when compared to SF isolated hMSCs (FIG. 9E).

Moreover, in FIGS. 9A-9F, the biologic characterization of hMSCs isolated from BM under different isolation procedures was assayed as: FIG. 9A shows Proliferation of hMSCs calculated as PD/day from passage 1 to 2, donor and isolation procedure dependent. FIG. 9B shows Proliferation average. FIG. 9C shows Chondrogenic potential, percentage of formed nodules, donor and isolation procedure dependent. FIG. 9H shows Chondrogenesis average. FIG. 9I shows Adipogenic potential, quantification of Oil red O staining relative to 100% Oil red O staining solution, donor and isolation procedure dependent. FIG. 9J shows Adipogenesis average. Values are represented as mean±standard deviation of at least three independent experiments (n≧3). Statistically significant differences were found with ***p<0.001 and **p<0.01.

FIGS. 11A1-A2, 11B, 11C, 11D1-D2, 11E, and 11F show surface marker expression (in percentage) represented as a bar plot; each bar stands as the average over the percentage of surface markers obtained from three donors. Selected sets of cell surface markers expressed positive on hMSC. All the other investigated sets were expressed negative for both conditions, and are therefore not shown. No statistically significant differences were found between the two conditions.

The expression of CD271, CD34, CD14, CD79a, CD45 and HLA-DR was absent in all conditions regardless of the isolation procedure, while no significant differences where observed in the expression of CD90, CD105, CD73 and CD146 between the isolation conditions. However, a trend in higher expression of CD105, CD73 and CD146 was observed in the hMSCs isolated in SF media (FIGS. 11A1-11F). The high standard deviation is the result of inter-donor variation.

### Analysis

The human body has an extensive capacity to regenerate bone tissue after trauma, however large defects cannot be restored without intervention and often lead to nonunion. Long bone fracture repair has been extensively studied at both the clinical as well as the fundamental research level, however little is known about the differences in fracture repair between the femur and the tibia. Therefore the present study assessed the pool and biological functions of BM-derived hMSCs in the lower limbs, such as the ilium, proximal femur, distal femur and proximal tibia. Additionally, we investigated methods to prime BM-derived hMSCs for reimplantation at the fracture site, to ease their homing and commitment towards faster bone regeneration, as it has been already shown that a reduced pool of proliferative and multipotent hMSCs are present at low healing fractures (See, Seebach C, Henrich D, Tewksbury R, Wilhelm K, Marzi I, Number and proliferative capacity of human mesenchymal stem cells are modulated positively in multiple trauma patients and negatively in atrophic nonunions, Calcified tissue international 2007; 80(4): 294-300).

In the present study we showed that the pool of BM-derived hMSCs differ with respect to the BM aspiration location. We found that after 14 days the number of hMSCs isolated from the ilium and proximal femur was higher (FIGS. 11A1-F) and that they showed higher self-renewal and osteogenic differentiation potential (FIGS. 7D and 7F) in comparison to hMSCs isolated from the distal femur and proximal tibia, with the later showing higher adipogenic potential. These findings correspond to the macroscopic appearance of the BM, with red BM found in the ilium and proximal femur, suggesting an active participation to hematopoiesis, and yellow BM found in distal femur and proximal tibia, containing a considerable admixture of fat. During aging read marrow is replaced by yellow marrow and this change in the marrow compartment might differ in fracture repair rates (See, Malkiewicz A, Dziedzic M., Bone marrow reconversion—imaging of physiological changes in bone marrow, Polish journal of radiology/Polish Medical Society of Radiology 2012; 77(4): 45-50). In this context, we understand that the differences in BFH rate between femur and tibia are the result of insufficient amounts of hMSCs present at the fractured site, as well as their poor self-renewal and osteogenic ability. To improve the current clinical treatment strategy, we have invented the present isolation of BM from the ilium and other locations in bone and its delivery in tibial fractures to enhance bone healing.

In the process of quantifying the concentration of MNCs with regard to the aspirated BM volume, we found that 10 mL of BM yields the highest MNC concentration. Higher BM volumes yielded low concentrations of MNCs, due to the dilution with peripheral blood during aspiration, while lower BM volumes also yielded lower concentrations of MNCs, as described by Fennema E M et al. (See, Fennema E M, Renard A J, Leusink A, van Blitterswijk C A, de Boer J., The effect of bone marrow aspiration strategy on the yield and quality of human mesenchymal stem cells, Acta orthopaedica 2009; 80(5): 618-21). Interestingly, in both studies the same averaged concentration of MNCs (2.6×10⁷ MNCs/mL) was found at 10 mL aspirated BM; thus resulting in the prior art encouraging the surgeons to limit the aspirated BM volume from the ilium to 10 mL.

To increase the contribution of cells to bone repair, a new dynamic vision emerged in tissue regeneration, focusing on rhythms and oscillatory patterns capable of orchestrating cell fate decision. The use of physical energy, such as ultrasound vibration, has been shown to affect the cell fate and increase the rate of bone repair (see, e.g., Choi W H, Choi B H, Min B H, Park S R., Low-intensity ultrasound increased colony forming unit-fibroblasts of mesenchymal stem cells during primary culture, Tissue engineering Part C, Methods 2011; 17(5): 517-26), however at insufficient cell number the therapy has been rather inefficient (tibia) (see, Fong K, Truong V, Foote C J, et al., Predictors of nonunion and reoperation in patients with fractures of the tibia: an observational study, BMC musculoskeletal disorders 2013; 14: 103).

Therefore, a different approach is provided for: delivery of acoustic stimulated BM from the ilium (rich in hMSCs) at the fracture site. Based on a previous study by Ridgway J. et al., where acoustic vibration was used to separate cells from BM suspension by trapping the cells in the pressure node planes of the standing wave and reducing the volume, an increase in CFU-Ob potential was observed in the processed BM (see, Ridgway J, Butcher A, Chen P S, Horner A, Curran S., Novel technology to provide an enriched therapeutic cell concentrate from bone marrow aspirate, Biotechnology progress 2010; 26(6): 1741-8). We understand that this increase was not only the result in BM volume reduction, but also a change in cell fate. To test this we selected two different frequencies in the range of acoustic vibration, 300 and 500 Hz, and two time points of 5 and 10 minutes. The results obtained showed a trend towards an increased self-renewal and a shift towards osteogenic and chondrogenic but not adipogenic differentiation in acoustic stimulated BM, perhaps suggesting that hMSCs may sense the acoustic vibratory frequencies. However, the long expansion period necessary to obtain sufficient cell numbers to perform the assays eventually led to a decrease in the multilineage potential, as cell potential is known to diminish with increased in vitro culture time (see, e.g., Li H, Fan X, Kovi R C, et al., Spontaneous expression of embryonic factors and p53 point mutations in aged mesenchymal stem cells: a model of age-related tumorigenesis in mice, Cancer research 2007; 67(22): 10889-98). In addition, we understand that the decrease in positive hMSC surface markers in acoustic stimulated BM is a result of integrin reorganization (cellular mechanoreceptor on the cell surface), followed by surface marker reorganization (see, e.g., Yang R S, Lin W L, Chen Y Z, et al., Regulation by ultrasound treatment on the integrin expression and differentiation of osteoblasts, Bone 2005; 36(2): 276-83) and change in cell fate.

The techniques herein describe the use of acoustic energy applied directly on BM (and not on cells) paving the way to its implementation into a one-step surgical procedure for bone repair. While acoustic sound vibration focuses on changing the phenotype of the cells, variation of the initial hMSCs isolation conditions focuses on the selection of a defined cell pool. Isolation of hMSCs in SF media selects a pro-chondrogenic cell population, which correlates with well-established differentiation protocols, where chondrogenic differentiation of cells is induced in serum free media (see, e.g., Mackay A M, Beck S C, Murphy J M, Barry F P, Chichester C O, Pittenger M F, Chondrogenic differentiation of cultured human mesenchymal stem cells from marrow, Tissue Eng 1998; 4(4): 415-28). The use of serum free isolated hMSCs with pro-chondrogenic potential can be of help in soft-callus formation after a bone fracture and where chondrocytes play a key role. Additionally, isolation of hMSCs at reduced MNC plating number selects a pro-adipogenic cell population. These findings underline the importance discovered and disclosed herein of carefully selecting the right isolation procedure for the right application.

Overall, the results show that novel approaches to bone fracture healing have been developed based on an understanding of bone marrow cell biology. Based on these results, poor BFH in the tibia may be the result of insufficient cell numbers, as well as their poor osteogenic potential. Based on this understanding, aspiration of BM from the ilium (and other remote sites of bone) and its delivery into the tibia accelerates fracture healing. Moreover, these techniques may be applied to at least two new therapeutic approaches for BFH: acoustic stimulation of BM and use of preselected pro-chondrogenic hMSC pool for delivery at the fracture site.

In non-limiting summary, therefore, large bone marrow volumes can be aspirated from the ilium with the highest concentration in mononuclear cells at 10 mL. The more proximal the bone marrow aspiration location, the larger the volume of bone marrow, the higher the content in mononuclear cells/hMSCs and the higher the self-renewal and osteogenic potential. The adipogenic differentiation potential of the isolated hMSCs may not apply. Further, acoustic stimulation of bone marrow, as well as the isolation of hMSCs in the absence of fetal bovine serum and culturing at lower densities than previously done, increases respectively the osteogenic and chondrogenic potential of the cells. These increases assist the healing of bone fractures.

Bone marrow aspirated from different locations of the lower limbs have different properties, potentially explaining the differences in bone fracture healing between the tibia and the femur. Furthermore, two new priming (activating) methods capable of enhancing bone fracture healing are provided.

While the subject matter has been described with respect to a limited number of embodiments, those skilled in the art, having benefit of this disclosure, will appreciate that other embodiments can be devised which do not depart from the scope of the subject matter as disclosed herein. Accordingly, the scope of the invention should be limited only by the attached claims.

The above description is illustrative of the preferred embodiment and many modifications may be made by those skilled in the art without departing from the invention whose scope is to be determined from the literal and equivalent scope of the claims that follow.

While the embodiments are described with reference to various implementations and exploitations, it will be understood that these embodiments are illustrative and that the scope of the inventive subject matter is not limited to them. Many variations, modifications, additions and improvements are possible, such as alterations to the site of harvesting of the BM, the amount of BM harvested, primed, and used, variations in the level of acoustic energy exposed to the cells, the volume exposed, the time of exposure, the device(s) used for exposure, and variations in the plating of the cells such as the components of the media, density of the cells, types of plates and conditions of culture, length of culture, and for non-limiting example, length of culture.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

What is claimed is:

1. A method of using human mesenchymal stromal cells (hMSC) from bone marrow regions distal from a bone fracture for encouraging healing the bone fracture, comprising:
accessing bone marrow comprising hMSCs in region distal to a bone fracture;
aspirating bone marrow from the accessed bone marrow region;
activating the hMSC from the aspirated bone marrow; and
administering the activated hMSCs to the site of the bone fracture, wherein the activated hMSCs enhance healing of the bone fracture.

2. The method of claim 1, wherein enhancing is performed by acoustic energy stimulation.

3. The method of claim 1, wherein enhancing is performed by an acoustic stimulation device.

4. The method of claim 3, wherein the acoustic stimulation device exposes the bone marrow aspirate to 300 Hz of acoustic energy.

5. The method of claim 3, wherein the acoustic stimulation device exposes the bone marrow aspirate to an acoustic energy standing wave.

6. The method of claim 3 wherein the acoustic stimulating device comprises a chamber for holding the bone marrow aspirate, and a speaker for generating acoustic energy.

7. An isolated human mesenchymal stromal cell (hMSC) isolated from bone marrow aspirate obtained from a site distal to a bone fracture and activated in vitro for enhanced healing properties for bone fracture healing comprising at least one of the properties of enhanced bone fracture healing selected from the group consisting of self-regeneration potential, multilineage potential, proliferation potential, and surface marker expression.

8. The isolated hMSC of claim 7, wherein the enhanced bone fracture healing is from enhanced self-regeneration potential of the isolated hMSC.

9. The isolated hMSC of claim 7, wherein the enhanced bone fracture healing is from enhanced multilineage potential of the isolated hMSC.

10. The isolated hMSC of claim 7, wherein the enhanced bone fracture healing is related to hMSC expressing certain cell markers bound by at least one antibody selected from the group consisting of CD90, CD73, CD146, CD105, CD271, CD34, CD14, CD79a, HLA-DR, and CD45.

11. The isolated hMSC of claim 7, wherein the enhanced bone fracture healing is from exposing the aspirated bone marrow to acoustic energy.

12. The isolated hMSC of claim 11, wherein the enhanced bone fracture healing is performed by an acoustic stimulation device.

13. The isolated hMSC of claim 12, wherein the enhanced bone fracture healing is performed by an acoustic stimulation device that exposes the bone marrow aspirate to 300 Hz of acoustic energy.

14. The method of claim 12, wherein the enhanced bone fracture healing is performed by an acoustic stimulation device exposing the bone marrow aspirate to an acoustic energy standing wave.

15. A system for treating bone fractures by using human mesenchymal stromal cells isolated from regions of bone marrow distal to a bone fracture, comprising:
an acoustic energy stimulator for exposing a bone marrow aspirate aspirated from a bone marrow from a region distal to a bone fracture, the aspirated cells comprising human mesenchymal stromal cells (hMSC); and
a source of acoustic energy associated with the acoustic energy stimulator.

16. The system of claim 15, wherein the source of acoustic energy is a speaker.

17. The system of claim 15, wherein the acoustic energy stimulator has a tube for draining the hMSCs after they are exposed to acoustic energy.

15. The system of claim 15, wherein the acoustic energy stimulator exposes the bone marrow aspirate to 300 Hz.

16. The system of claim 15, wherein the acoustic energy stimulator exposes the bone marrow aspirate to an acoustic standing wave.
